(19) United States
(12) Patent Application Publication
(10) Pub. No.: US 2011/0052693 A1
(43) Pub. Date: Mar. 3, 2011

(54) METHOD FOR PRODUCING ARTIFICIAL SKIN

(76) Inventors: Bunsho Kao, Tokyo (JP); Yoshiaki Hosaka, Tokyo (JP); Ayumi Iijima, Tokyo (JP)

(21) Appl. No.: 12/867,357

(22) PCT Filed: Sep. 19, 2008
(86) PCT No.: PCT/JP2008/067024

§ 371 Date: Aug. 12, 2010

(30) Foreign Application Priority Data
Feb. 29, 2008 (JP) 2008-049337

(51) Int. Cl. A61K 9/14; C12N 5/00; A61K 35/36; A61P 17/02

(52) U.S. Cl. 424/484; 435/373; 424/93.7

(57) ABSTRACT

An object of the invention is to provide artificial skin that does not contain any animal-derived material or pathogen and has excellent biocompatibility. The invention provides, as a solution means, a method for producing artificial skin comprising the steps of: (A) forming a dermal layer by solidifying a mixture of dermal fibroblasts and a peptide hydrogel having a fibrous structure; and (B) forming an epidermal layer by seeding skin keratinocytes onto the dermal layer obtained in Step (A), and culturing the epidermal keratinocytes.

### TECHNICAL FIELD

The present invention relates to a method for readily producing safe hybrid artificial skin.

### BACKGROUND ART

Ex-vivo culture of cells collected from living tissues is now commonly practiced using culture flasks in the laboratory. This has revealed the characteristics of various cells, providing a deeper understanding of our tissues and organs, thereby contributing to medical science. Recently, however, the fact that it is difficult to completely reproduce what is happening in vivo by two-dimensional culture of only cells has been recognized.

Some cells spreading two-dimensionally in the culture flask adhere to the flask, some adhere to neighboring cells, and others are directly exposed to the culture medium. Therefore, nutrients, various growth factors, and cytokines contained in the culture medium directly act upon individual cells. In-vivo cells are arranged three-dimensionally, with an extracellular matrix filling between the cells; therefore, nutrients, various growth factors, and cytokines spread by diffusion, as well as by signaling between cells, and signaling between cells and the extracellular matrix. In particular, the extracellular matrix has recently been recognized as being important, and also indicated as playing an important role in the differentiation of stem cells (Non-Patent Documents 1 and 2).

The goal of tissue engineering and regenerative medicine is to repair a patient's functions by using living cells, tissues, or organs that will ultimately become incorporated into the patient's body (Non-Patent Document 3). For this reason, in tissue engineering and regenerative medicine, three-dimensional, tissue-like structures have been constructed using scaffolds for cultured cells. Ideally, a scaffold should meet, for example, the following conditions: 1) it has a basic structure that can be easily designed and modified; 2) it can control in-vivo degradation; 3) it is non-cytotoxic; 4) it has properties that specifically promote or inhibit the relationship between cells and a substance; 5) it rarely elicits an immunological reaction or inflammatory reaction; 6) it can be easily produced in large amounts at low cost; and 7) it has a physiological affinity (Non-Patent Document 4).

Among studies ongoing in the field of regenerative medicine, research into skin substitutes utilizing skin cells has shown significant advances. Examples of scaffolds that have been developed for artificial skin substitutes include a scaffold formed using a net made of a bioresorbable synthetic polymer; a scaffold formed by attaching a nylon net to a silicon film; a scaffold having a two-layered structure of a collagen sponge and a silicon sheet (Non-Patent Document 5); a scaffold formed using an atelocollagen sponge made into a sheet; a scaffold formed by matching collagen sponges having different pore sizes (Non-Patent Document 6); and acellular dermal matrices (ADM) formed using fibrin glue or allogeneic skin that has been made cell-free (Non-Patent Documents 7 and 8).

However, hybrid artificial skin containing living fibroblasts and epidermal keratinocytes, which is currently being researched and developed, often uses an animal-derived substance as a scaffold, thus involving a potential risk of unknown infections. Some known scaffolds for artificial skin substitutes use animal-derived substances such as cow, pig, or rat-derived collagen, fibrin glue, and allogeneic dermal matrices (Non-Patent Documents 9 and 10). The administration or transplantation of a substance derived from an animal or another person's tissue, even if it appears to be safe at the time, poses a potential risk of unknown infections, not to mention cases of HIV infections caused by the administration of blood products to leukemia patients, and the development of Creutzfeld-Jacob disease due to the use of imported dried dura mater.

In order to popularize regenerative medicine, such as using artificial skin as a general therapy, it is necessary to replace natural materials, which are non-uniform, have limited functions, and pose a risk of infections, with synthetic materials that are safe and normalized, and can incorporate functions that are readily used.

### Non-Patent Document 1: Engler, A J et al., Cell 2006, Aug 25; 126 (4): 677-689

### Non-Patent Document 2: Narmoneva, D A et al., Biomaterials 2005, Aug; 26 (23): 4837-4846.

### Non-Patent Document 3: Vacanti, J P et al., Lancet 1999, Jul; 354 Suppl 1: SI32-34.

Non-Patent Document 4: Holmes, T C et al., Trends in Biotechnology 2002, Jan; 20(1): 16-21.

### Non-Patent Document 5: Yannas, I V et al., Journal of Biomedical Materials Research 1980, Jan; 14(1): 65-81.

### Non-Patent Document 6: Morikawa, Noriyuki et al., Journal of the Japanese Association of Regenerative Dentistry, Vol. 3 (1): 12-22, 2005

### Non-Patent Document 7: Ghosh, M M et al., Annals of Plastic Surgery 1997, Oct; 39 (4): 390-404.

Non-Patent Document 8: Yamaguchi, Ryo et al., Japanese journal of Burn Injuries; 30 (3): 152-160, 2004.

### Non-Patent Document 9: Bokhari, M A et al., Biomaterials 2005, Sep; 26 (25): 5198-5208.

### Non-Patent Document 10: Bell, E et al., Science (New York, N.Y. 1981, Mar 6; 211 (4486): 1052-1054

### DISCLOSURE OF THE INVENTION

### Problems to be Solved By the Invention

An object of the invention is to provide artificial skin that does not contain any animal-derived material or pathogen and has excellent biocompatibility, by using a novel method for producing artificial skin.

### Means For Solving the Problems

The inventors conducted extensive research to solve the above-mentioned object. Consequently, they found that safe artificial skin can be produced by preparing cultured dermis obtained by three-dimensional culture of human fibroblasts, using a peptide hydrogel that poses no risk of unknown infections as a scaffold; and by preparing cultured skin by additionally forming an epidermal layer on the cultured dermis using human epidermal keratinocytes. The invention was accomplished based on this finding.

Specifically, the invention includes the following features.

Item 1. A method for producing artificial skin comprising the steps of:

(A) forming a dermal layer by solidifying a mixture of dermal fibroblasts and a peptide hydrogel having a fibrous structure; and

(B) forming an epidermal layer by seeding epidermal keratinocytes onto the dermal layer obtained in Step (A), and culturing the epidermal keratinocytes.

Item 2. The method according to Item 1, wherein the peptide hydrogel is a synthetic matrix comprising 3 to 0.1% (w/v) of amino acids and 97 to 99.9% (w/v) of water.

Item 3. The method according to Item 1, wherein the peptide hydrogel is a synthetic matrix comprising 1 to 0.1% (w/v) of amino acids and 99 to 99.9% (w/v) of water.

Item 4. The method according to Item 2 or 3, wherein the peptide of the peptide hydrogel is a peptide comprising 12 to 30 amino acids and having alternating hydrophobic and hydrophilic side chains, or a modified product of the peptide.

Item 5. The method according to Item 4, wherein the amino acids are three or more types of amino acids selected from the group consisting of arginine, aspartic acid, alanine, lysine, leucine, proline, threonine, and valine.

Item 6. The method according to Item 4, wherein the amino acids consist of arginine, asparagine, and alanine.

Item 7. The method according to Item 4, wherein the peptide of the peptide hydrogel consists of an amino acid sequence represented by any of SEQ ID NOS. 1 to 6.

Item 8. Artificial skin produced by the method according to any one of Items 1 to 7.

Item 9. The artificial skin according to Item 8, which is used for skin grafting.

### EFFECTS OF THE INVENTION

According to the method for producing artificial skin of the invention, artificial skin that does not contain any animal-derived material or pathogen and has excellent biocompatibility can be produced. Moreover, in the method of the invention, a culture medium that is free of any animal-derived component, such as Fetal Bovine Serum (FBS), is used together with the above-mentioned scaffold, thereby enabling the production of a hybrid artificial skin material wherein neither the culture medium nor scaffold contain components derived from an animal or another person's tissue.

Further, in the method of the invention, the peptide hydrogel used as a scaffold can be readily mixed with cells and bioactive molecules (growth factors) during self-assembly, and is also unlikely to induce an immunological reaction because of its low molecular weight. Furthermore, the peptide hydrogel has a physiological affinity for tissues, and has no cytotoxic effects because it is degraded to amino acids, which are inherently present in large amounts within tissues.

Furthermore, in known methods for producing artificial skin using collagen gel, the collagen gel remains even after long-term culture; by contrast, in the method of the invention, the peptide hydrogel used as a scaffold for grafting is degraded after the passage of a necessary period of time, and therefore, does not remain in tissues. This provides the advantage of promoting the migration, proliferation, and differentiation of the cultured cells. In summary, the method of the invention is useful for growing skin in vivo, and the artificial skin produced by the method of the invention is particularly suitable for clinical graft applications.

The artificial skin produced by the method of the invention uses a synthetic material consisting of amino acids as a scaffold. This eliminates the costs that are incurred from removing potentially contained pathogens when using an animal-derived material as a scaffold; therefore, the artificial skin of the invention can be prepared at low cost.

Furthermore, excluding cells, the artificial skin produced by the method of the invention uses synthetic materials, so that it can be produced in large amounts with uniform quality. Furthermore, the artificial skin produced by the method of the invention contains no bioactive molecules (growth factors), which are endogenous substances that become problematic when artificial skin contains natural materials.

### BRIEF DESCRIPTION OF THE DRAWINGS

FIG. 1 shows the peptide hydrogel (PuraMatrix (registered trademark)) used in Example 1.

FIG. 2 shows a schematic diagram of the method of the invention. 1: The peptide hydrogel solidified due to a change in pH. Because the peptide hydrogel solution has a pH of 3, fibroblasts were temporarily exposed to strong acidity during mixing and were lost. Viability of the fibroblast was higher on the surface that was more rapidly neutralized. 2: Neonatal skin keratinocytes were seeded onto the resulting cultured dermis to prepare an epidermal layer. 3: Cornification of keratinocytes was promoted by exposing the epidermis of the resulting epidermal layer to outside air. After the preparation of the dermal layer, a culture was performed for 5 weeks.

FIG. 3 shows micrographs (at magnifications of 20 and 100 times) of H&E stained tissue specimens of the cultured dermis prepared in the Examples after 1, 2, 4, and 5 weeks of the culture. After 4 weeks, degradation of the peptide and a decrease in strength were observed.

FIG. 4 shows micrographs (at magnifications of 20, 100, and 400 times) of the H&E stained epidermal layer after 3 weeks of preparing the dermal layer (1 week after the preparation of the epidermal layer). An observation of the micrograph at a magnification of 20 times indicates that the epidermal layer formed over the entire specimen, but had partially peeled off the dermal layer (after 4 weeks, the epidermal layer of the specimens had completely peeled off). An observation of the micrograph at a magnification of 100 times indicates that fibroblasts were substantially evenly distributed over the entire dermal layer. Moreover, the septate structure was partially collapsed, and about 3 to 5 layers of stratified keratinocytes were observed in the epidermis.

FIG. 5 is a graph showing the cell counts of fibroblasts until week 5 of the culture (measured by the MTS assay).

FIG. 6 is a graph showing increases in the quantity of human type I collagen in the cultured dermis (for 5 weeks).

FIG. 7 is a graph showing increases in the quantity of human type I collagen in the culture media for culturing dermis (for 5 weeks).

FIG. 8 shows micrographs (each at magnifications of 20, 100, and 400 times) of human type I collagen staining of fibroblasts in the cultured dermis; and micrographs (each at magnifications of 20, 100, and 400 times) of laminin staining of the basal membrane in the cultured skin. Collagen stained most positively in regions contacting the epidermis of the dermal layer.

FIG. 9 shows micrographs each of fibronectin staining and human type IV collagen staining of the basal membrane in the cultured skin (at a magnification of 400 times). Partial staining indicates that the basal membrane is present, although not completely.

FIG. 10 shows micrographs (at magnifications of 40 and 200 times) of antibody staining of keratinocytes in the cultured skin. The micrographs in the upper section show nuclear transcription factor p63 staining for cells that are undifferentiated and capable of division; the micrographs in the middle section show cytokeratin 1/10/11 staining for differentiated keratinocytes (prickle cells); and the micrographs in the lower section show cytokeratin 14 staining for basal cells. The keratinocytes were positively stained with nuclear transcription factor p63 and cytokeratin 1/10/11, and negatively stained with cytokeratin 14; therefore, the artificial skin of the invention was found to mostly contain basal cells that were undifferentiated and highly capable of division.

### BEST MODE FOR CARRYING OUT THE INVENTION

In the invention, various types of commercially available cell strains can be used as fibroblasts (in particular, dermis-derived fibroblasts) and keratinocytes. Fibroblasts and keratinocytes may also be prepared by culturing those derived from animals, in particular, human skin. Especially for use in clinical skin grafting, it is preferable to culture fibroblasts and keratinocytes derived from a patient's own skin, excluding the portion that requires skin grafting.

The peptide hydrogel used in the invention is not limited as long as it has a fibrous structure, and contains amino acids that are not derived from animals as principal components. In one specific embodiment, the peptide hydrogel is, for example, a synthetic peptide (a synthetic matrix) containing 3 to 0.1% (w/v) of amino acids and 97 to 99.9% (w/v) of water; and preferably, a synthetic peptide (a synthetic matrix) containing 1 to 0.1% (w/v) of amino acids and 99 to 99.9% (w/v) of water.

In a preferred embodiment, the peptide forming the peptide hydrogel used in the invention is, for example, a peptide containing 12 to 30 amino acids and having alternating hydrophobic and hydrophilic side chains.

The amino acids forming the peptide may be three or more types of amino acids selected from the group consisting of arginine, aspartic acid, alanine, lysine, leucine, proline, threonine, and valine. Possible combinations of amino acids include a combination of arginine, asparagine, and alanine; a combination of valine, lysine, proline, and threonine; and a combination of lysine, leucine, and aspartic acid. Among the above, arginine, asparagine, and alanine, which are standard amino acids, are preferable as the amino acids forming the peptide in the peptide hydrogel according to the invention. The peptide may also be modified.

Examples of preferable peptides include those consisting of the amino acid sequences represented by SEQ ID NOS. 1 to 3. Examples of modified peptides include those consisting of the amino acid sequences represented by SEQ ID NOS. 4 to 6. In a further preferred embodiment, it is preferable to use a peptide hydrogel containing the peptide consisting of the amino acid sequence represented by SEQ ID NO. 1, the peptide hydrogel containing 3 to 0.1% (w/v) of amino acids and 97 to 99.9% (w/v) of water; it is most preferable to use a peptide hydrogel containing the same peptide, and containing 1 to 0.1% (w/v) of amino acids and 99 to 99.9% (w/v) of water.

The peptide hydrogel used in the invention forms a scaffold having a nanometer-scale fibrous structure in which the peptide is self-assembled due to a change in pH to form a β-sheet structure. This scaffold is a matrix having a highly purified peptide sequence that promotes cell adhesion, and forms a three-dimensional fibrous structure with an average pore size of 50 to 200 nm.

Peptide hydrogels disclosed in, for example, U.S. Pat. No. 5,670,483, as well as other commercially available peptide hydrogels, may be used as the peptide hydrogel of the invention. Alternatively, the peptide hydrogel used in the invention can be prepared according to known solid-phase synthesis or the like, using a peptide synthetizer.

The method for producing artificial skin of the invention includes the following steps:

(A) forming a dermal layer by solidifying a mixture of dermal fibroblasts and a peptide hydrogel having a fibrous structure; and

(B) forming an epidermal layer by seeding epidermal keratinocytes onto the dermal layer obtained in Step (A), and culturing the epidermal keratinocytes.

In Step (A), the above-mentioned peptide hydrogel is used as a scaffold on which the dermal layer of artificial skin is formed.

Step (A) of the method of the invention includes mixing the peptide hydrogel and fibroblasts, and solidifying the mixture, to form a dermal layer.

Specifically, fibroblasts are suspended in a 10% sucrose solution or the like at a concentration of about 3 to 30×10⁶ cells/cm³, and the suspension is mixed with an equal volume of 2% peptide hydrogel (approximately pH 3). The resulting mixture naturally solidifies because the pH is raised by mixing. A dermal layer is formed by culturing the solidified mixture.

Although the culture conditions are not limited, a culture is preferably performed for about 2 to 3 weeks, during which the dermal layer is soaked in a culture medium such as D-MEM medium at around 37° C. in 7.5% CO₂, and the culture medium is replaced every 2 to 3 days.

When artificial skin for use in clinical skin grafting is produced, a peptide, a drug, or the like that promotes cell migration, proliferation, and differentiation may be added to the peptide hydrogel, prior to mixing the peptide hydrogel and fibroblasts. Examples of such peptides or drugs include epidermal growth factor: EGF, insulin-like growth factor: IGF, transforming growth factor: TGF, nerve growth factor: NGF, brain-derived neurotrophic factor: BDNF, vesicular endothelial growth factor: VEGF, granulocyte-colony stimulating factor: G-CSF, granulocyte-macrophage-colony stimulating factor: GM-CSF, platelet-derived growth factor: PDGF, erythropoietin: EPO, thrombopoietin: TPO, basic fibroblast growth factor: bFGF or FGF2, and hepatocyte growth factor: HGF.

Step (B) of the method of the invention includes forming an epidermal layer by seeding epidermal keratinocytes onto the cultured dermal layer obtained in Step (A), followed by culturing. The artificial skin of the invention is thus obtained by culturing epidermal keratinocytes on the cultured dermal layer.

Preferably, keratinocytes are seeded onto the dermal layer at a concentration of about 3 to 6×10⁶ cells/cm³, and cultured for about 1 to 3 days at 37° C. in 5 to 7.5% CO₂ until complete cell adhesion is accomplished.

In order to promote the adhesion of the keratinocytes, prior to 3 to 7 days of seeding keratinocytes, fibroblasts may be additionally seeded onto the dermal layer at a concentration of about 3 to 30×10⁶ cells/cm³, thereby increasing the fibroblast density on the dermal layer surface.

Continued culturing of the artificial skin containing fibroblasts and keratinocytes allows the fibroblasts in the dermal layer to proliferate and differentiate to secrete collagen, thereby enhancing the strength of the dermal layer. After 3 weeks of continued culturing, the peptide hydrogel scaffold is gradually degraded. However, during the initial period of culturing the fibroblasts and keratinocytes, the peptide hydrogel is only partially degraded, and not completely degraded. Next, the medium is replaced with D-MEM medium supplemented with 10% FBS, KGM-2 medium, or a mixture containing equal volumes of these media, and a culture is performed for 1 to 2 weeks while adjusting the volume of the medium such that the keratinocytes are exposed to air. This also allows the keratinocytes in the epidermal layer to proliferate, thereby producing artificial skin containing 5 to 10 layers of stratified keratinocytes.

Note that the media (culture media) mentioned in the specification are merely illustrative of usable media, and are not intended to limit the media used in the method of the invention.

The method of the invention can be suitably used for producing, in particular, skin for grafting. When producing artificial skin for grafting, it is preferable to culture the cultured skin (containing the dermal layer and epidermal layer) for 3 to 4 weeks, and then graft the resulting skin having a residual peptide hydrogel content of 50 to 90%.

### EXAMPLES

The invention will be described in greater detail below, referring to examples; however, the invention is not limited by these examples.

### Example 1

### Method for Producing Artificial Skin

(1) Cell expansion (cell culture)

Neonatal human dermal fibroblasts (Lonza Walkersville, Walkersville, Md.) were subcultured 8 to 10 times in a culture flask using D-MEM medium (Lonza Walkersville, Walkersville, Md.) supplemented with 10% FBS (Invitrogen, Carlsbad, Calif.), and the cultured fibroblasts were used for the experiment. Neonatal human epidermal keratinocytes (Lonza Walkersville, Walkersville, Md.) were subcultured 4 or 5 times in a culture flask using KGM-2 medium (Lonza Walkersville, Walkersville, Md.), and the cultured keratinocytes were used for the experiment. Table 1 gives a summary of specific materials, reagents, and samples.

TABLE 1
Materials

Pura Matrix (Peptide hydrogel): 3D matrix Company
Normal human dermal fibroblasts (neonatal skin): Lonza Company
High glucose Dulbecco's Modified Eagle's Medium (D-MEM): Invitrogen
Corporation
Fetal Bovine Serum (FBS): Invitrogen Corporation
Penicillin: Invitrogen Corporation
Streptomycin: Invitrogen Corporation
Trypsin-EDTA: Invitrogen Corporation
Trypsin Neutralize Solution (TNS): Invitrogen Corporation
Multiwell (12 Well): Becton Dickinson Company
Cell Culture Inserts (3 μm): Becton Dickinson Company

(2) Preparation of Specimens

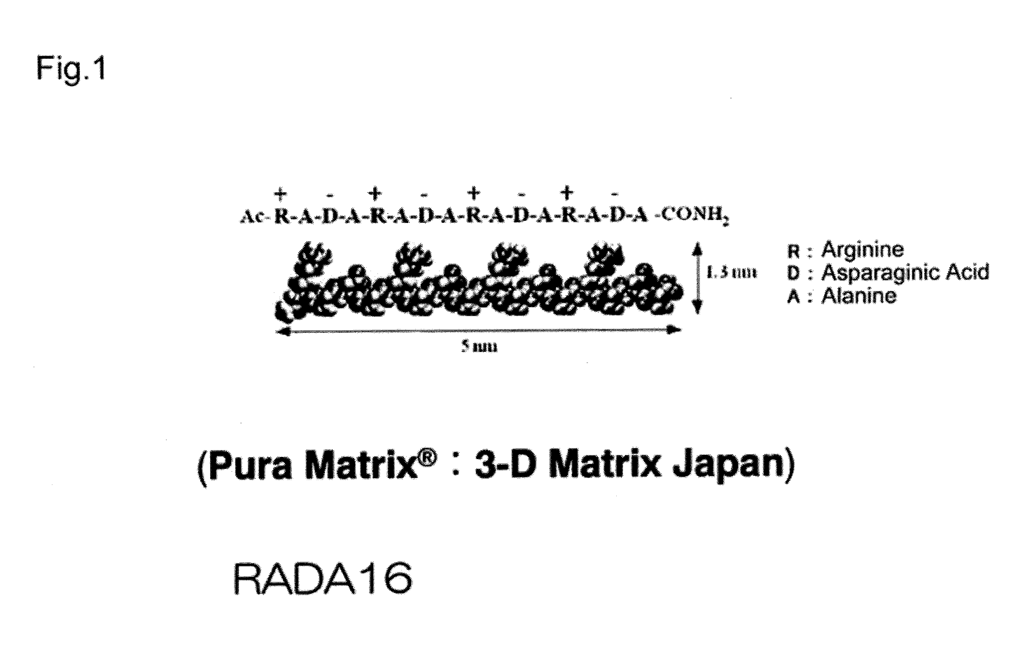

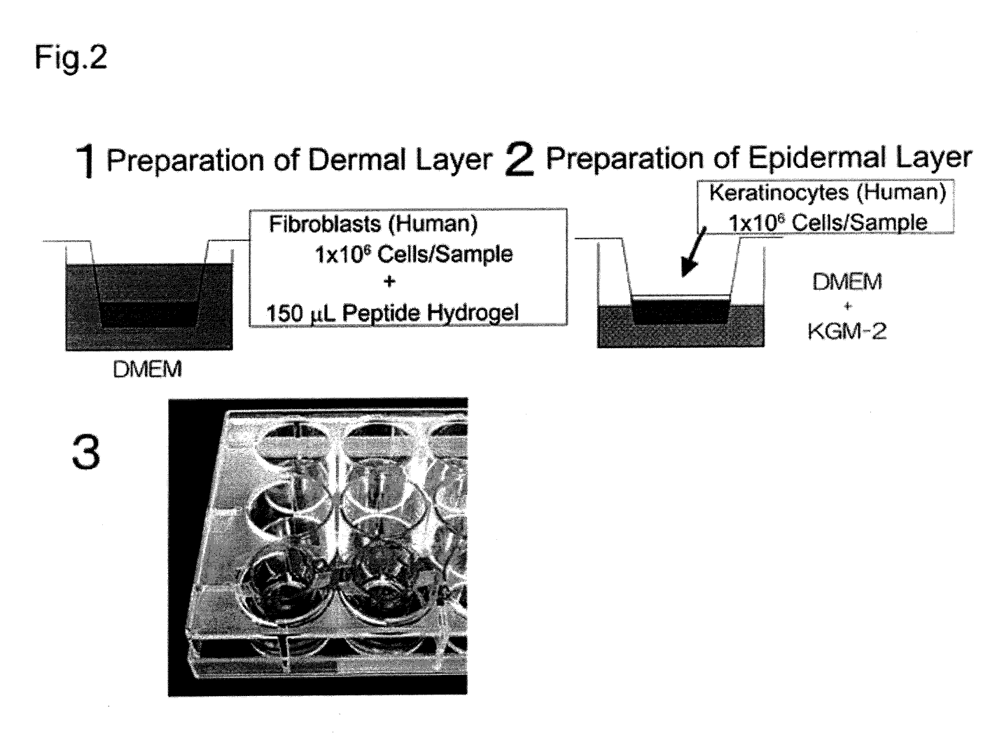

A 2% aqueous solution of peptide hydrogel RADA-16 (amino acid sequence: AcN-RARADADARARADADA-CNH₂; SEQ ID NO. 1; Pura Matrix (registered trademark) (FIG. 1); 3D Matrix Japan, Japan) (pH 3) was used as a scaffold for cultured dermis. 1×10⁶ human fibroblasts per sample were suspended in 150 μL of 10% sucrose solution, and the suspension was mixed with an equal volume of the 2% aqueous solution of peptide hydrogel RADA-16. The mixture was then immediately dispensed into cell culture inserts. The inserts were fixed in a 12-well plate, the periphery of each insert was filled with D-MEM medium, and the mixture of the fibroblasts and peptide hydrogel was allowed to solidify, thereby preparing a dermal layer (cultured dermis). The dermal layer kept in this state was cultured in an incubator at 37° C. in 7.5% CO₂. During the culture, the culture medium was replaced every 2 to 3 days. After 3 weeks of preparing the cultured dermis, the grown neonatal skin keratinocytes were seeded onto the cultured dermis, thereby preparing an epidermal layer (cultured skin). After the preparation of the cultured skin, the culture medium was replaced with a mixture of equal volumes of D-MEM supplemented with 10% FBS and KGM-2, and the culture was continued (FIG. 2).

(3) Histological and Immunochemical Analysis

Culturing of the cultured dermis was continued for 5 weeks after the preparation of the cultured dermis, and culturing of the cultured skin was continued for two weeks after the preparation of the cultured skin (for 5 weeks after the preparation of the cultured dermis). Every week, cultured specimens were fixed in 20% neutral formalin, dehydrated and embedded in low-temperature paraffin. Tissue specimens having a thickness of 6 μm were prepared, and subjected to H&E staining, and immunostaining. The specimens were then examined under a microscope.

Human type I collagen staining was performed as an index of the expression of the function of fibroblasts in the cultured dermis. Using a Ventana I-VIEW DAB universal kit, the cultured specimens were deparaffinized, washed with water and activated with protease. The specimens were labeled with anti-human collagen type I antibody (MP Biomedicals, Solon, Ohio) as the primary antibody, and nuclear staining was performed using hematoxylin.

Following the same procedure as above, laminin staining (Chemicon International, Temecula, Calif.), fibronectin staining (Santa Cruz Biotechnology, Santa Cruz, Calif.), and human type IV collagen staining (American Research Products, Belmont, Mass.) were performed as indices of the formation of the basal membrane in the cultured skin; and anti-nuclear transcription factor p63 antibody (Santa Cruz Biotechnology) staining, anti-cytokeratins 1/10/11 antibody (American Research Products) staining, and anti-cytokeratin 14 antibody (Progen Biotechnik, Germany) staining were performed as indices of the differentiation of epidermal keratinocytes.

(4) MTS Assay (Measurement of the Number of Cells)

The number of cells in cultured specimens of the cultured dermis was measured every week. Cells were counted using the CellTiter 96® AQueous One Solution Cell Proliferation Assay (Promega Corp., Madison, Wis.).

5 μL of the suspension of each disrupted specimen was mixed with 95 μL of D-MEM to give a total volume of 100 μL, and the mixture was placed in a 96-well plate as a sample. Following the manual, 20 μL of the reaction mixture was dispensed into each well, and reacted for 2 hours in an incubator. The absorbance was read at 490 nm using a plate reader. Cells were counted for six specimens every week. A Student's t-test was conducted to examine significant differences.

(5) Collagen Assay (Measurement of the Quantity of Human Type I Collagen)

Collagen was quantified in specimens of the cultured dermis that had been cultured for 5 weeks, as well as in their culture media. Quantification was conducted using a human type I collagen ELISA detection kit (AC Biotechnologies, Japan).

Following the manual, pepsin solution was added to each disrupted specimen and its culture medium, the mixture was shaken overnight at 4° C., and the pepsin was neutralized. The resulting mixture was used as a sample. 50 μL of a mixture of the sample and biotin-labeled collagen antibody solution was dispensed into each well of a microtiter plate on which collagen was immobilized, and reacted for 1 hour at room temperature. After washing the plate, 50 μL of HRP-labeled avidin solution was dispensed into each well, and further reacted for 1 hour at room temperature. After washing, 50 μL of TMB substrate was dispensed into each well, and further reacted for 15 minutes at room temperature. The absorbance was read at 450 nm using a plate reader. Collagen quantification was performed on six specimens every week, and a Student's t-test was conducted to examine significant differences.

### RESULTS

(1) H&E Staining of the Tissue Specimens

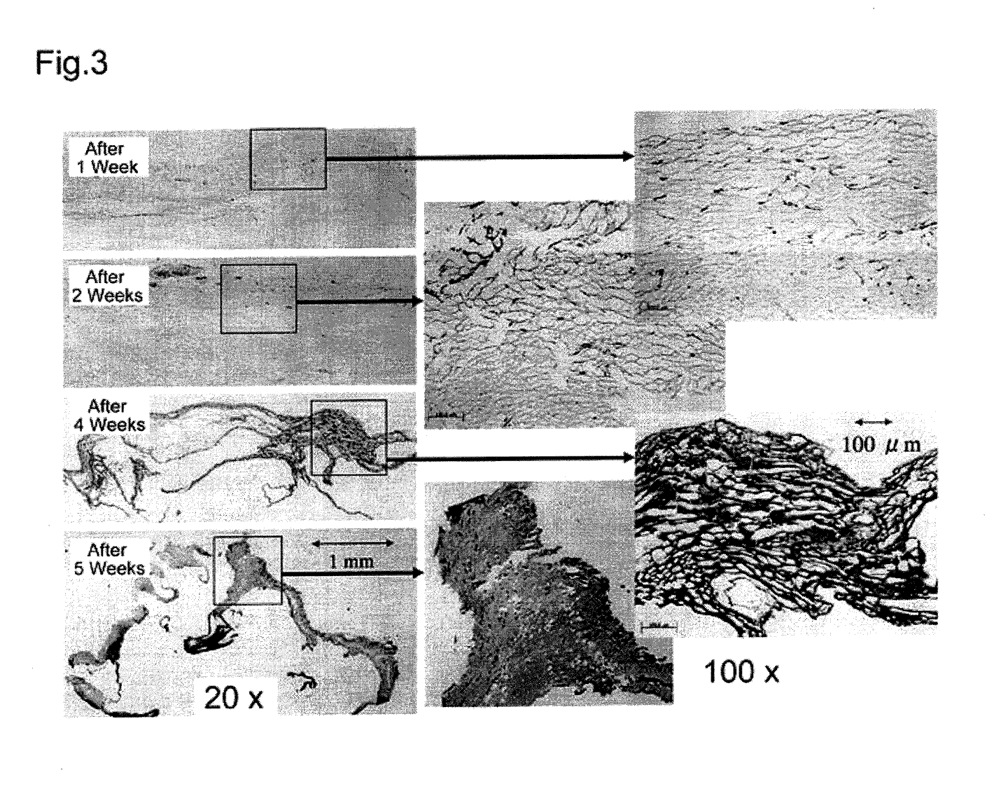

By performing H&E staining, cross sections of sponge-like three-dimensional structures of the peptide hydrogel were observed, showing the formation of dermis-like structures (FIG. 3; H&E stained cultured dermis at week 2; micrographs each at magnifications of 20 and 100 times). The initial dermal layer showed a foamy organization of the peptide hydrogel, and the presence of round fibroblasts in contact with septum of the peptide hydrogel. This demonstrates that a dermis-like tissue containing a three-dimensional culture of human fibroblasts was prepared. Fibroblasts proliferated as they formed cluster-like groups at various places within the septa, and proliferated with time as they changed into fusiform shapes along the septum. The septate structure partially collapsed with time (FIG. 3, week 5).

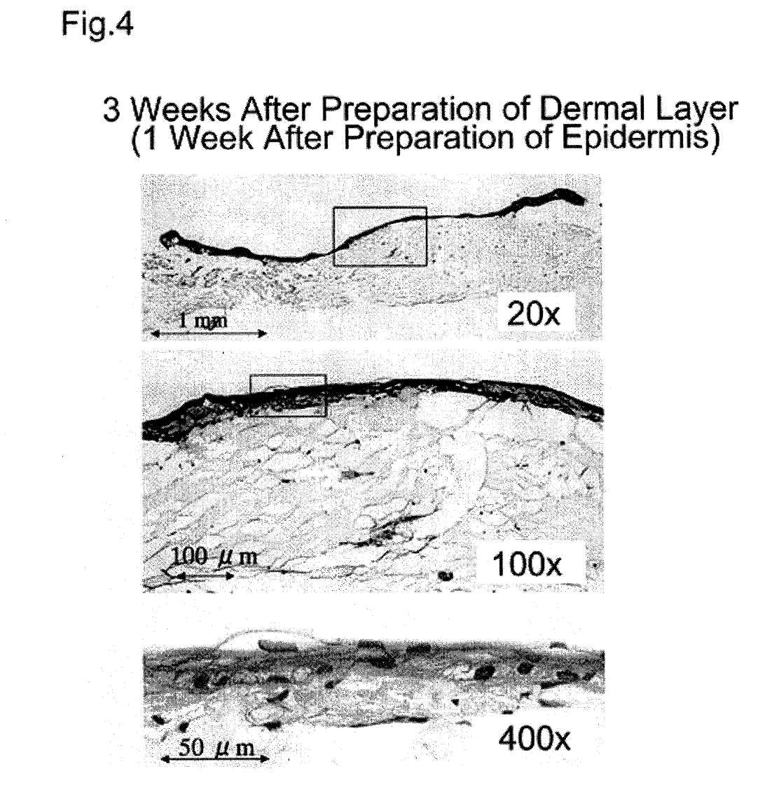

Epidermis containing stratified keratinocytes was formed on the cultured skin. An epidermal layer was formed over the entire specimen, but had partially ecfoliated from the dermal layer. The boundary between the dermal layer and epidermal layer was unclear and complicated. The epidermis was found to contain about 3 to 5 layers of stratified keratinocytes (FIG. 4).

(2) Measurement of the Number of Cells

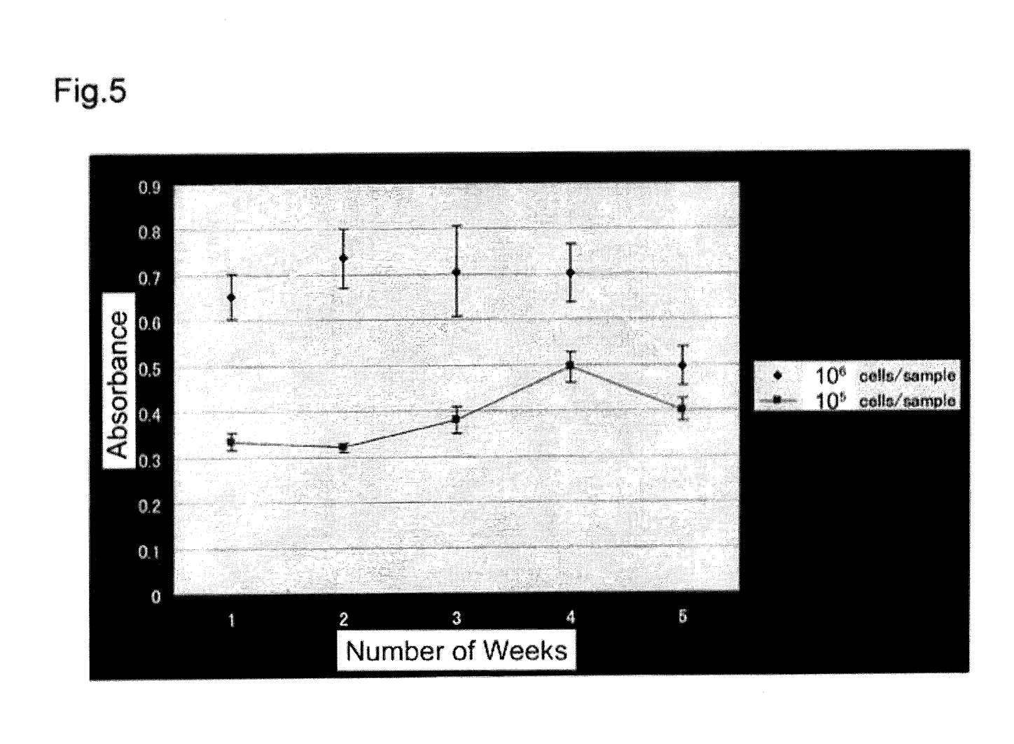

The number of cells in the cultured dermis showed a tendency to increase until week 2, but thereafter remained substantially constant until week 4, and then rapidly decreased at week 5. A Student's t-test was conducted for each set of 2 contiguous weeks; significant differences (p<0.05) were observed between the weeks 0 and 1, and the weeks 4 and 5 (FIG. 5).

(3) Collagen Quantification

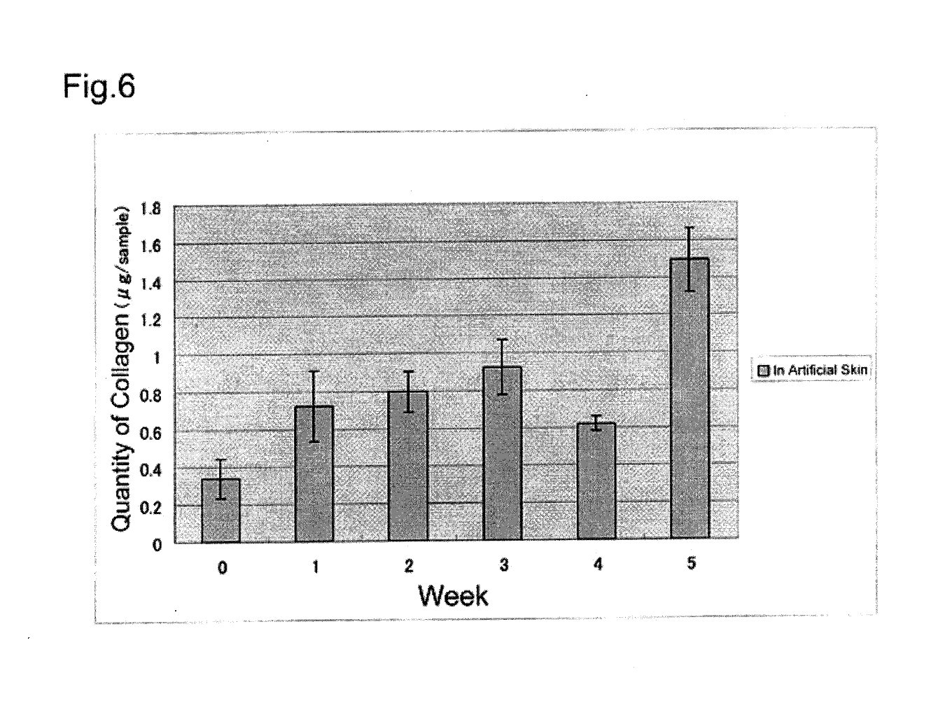

The quantity of collagen in the cultured skin specimens showed a tendency to increase until week 3, but no significant differences were observed; however, the quantity significantly increased at week 5. A Student's t-test was conducted for each set of 2 contiguous weeks; a significant difference (p<0.05) was observed between weeks 4 and 5 (FIG. 6).

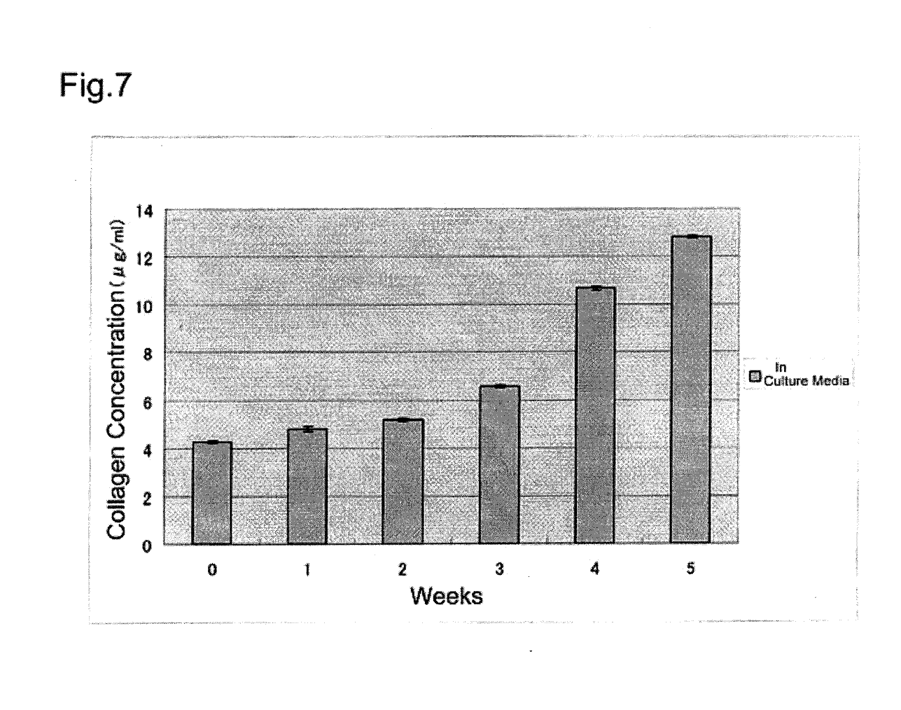

The quantity of collagen in the culture media did not change until week 2, but increased from weeks 3 to 5. A Student's t-test was conducted for each set of 2 contiguous weeks; significant differences were observed between weeks 2 and 3, weeks 3 and 4, and weeks 4 and 5 (FIG. 7).

(4) Immunohistochemistry

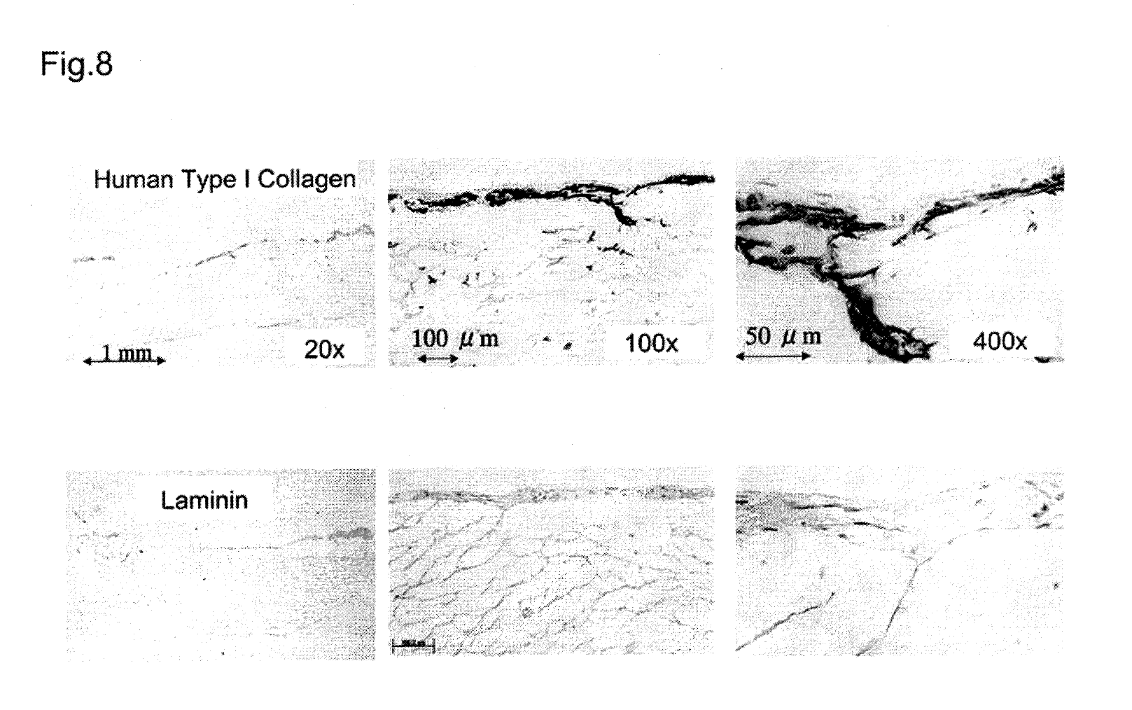

In immunohistochemical staining, positive staining with anti-human collagen type I antibody was observed around the cells within septum, demonstrating the presence of type I collagen secreted from the human fibroblasts. In particular, strong staining of human type I collagen remaining on the specimens surfaces was observed (FIG. 8; collagen).

In the artificial skin, human type I collagen stained most positively in regions contacting the epidermis of the dermal layer.

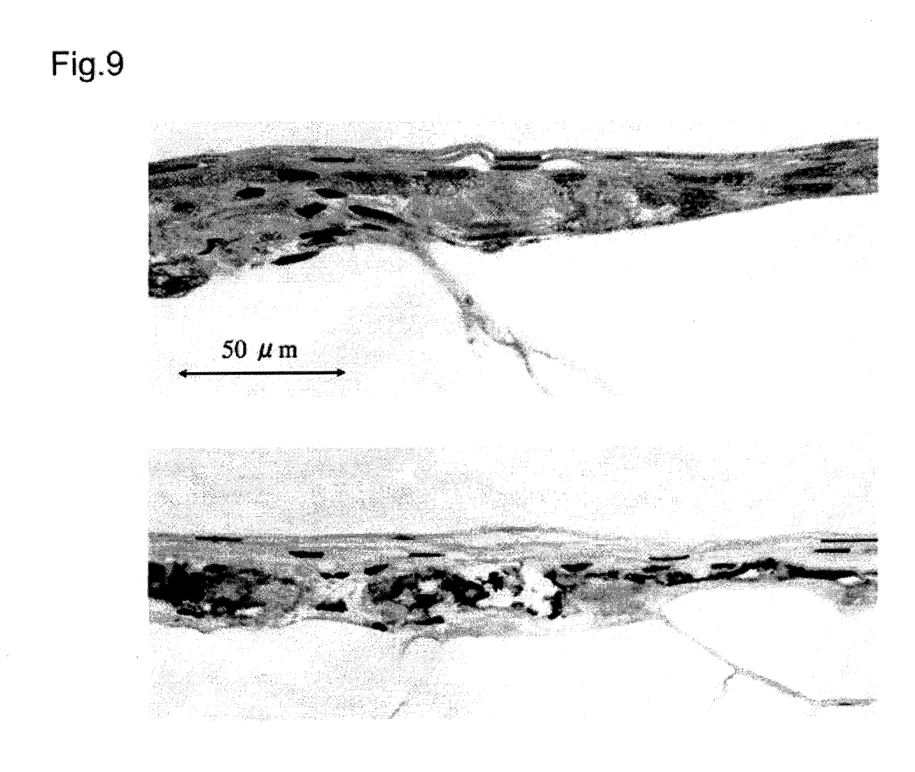

The presence of the basal membrane was indicated by partial staining with laminin, fibronectin, and human type IV collagen (FIG. 8: laminin, FIG. 9: fibronectin and human type IV collagen). The presence of the basal membrane stained with laminin was unclear (FIG. 8, laminin).

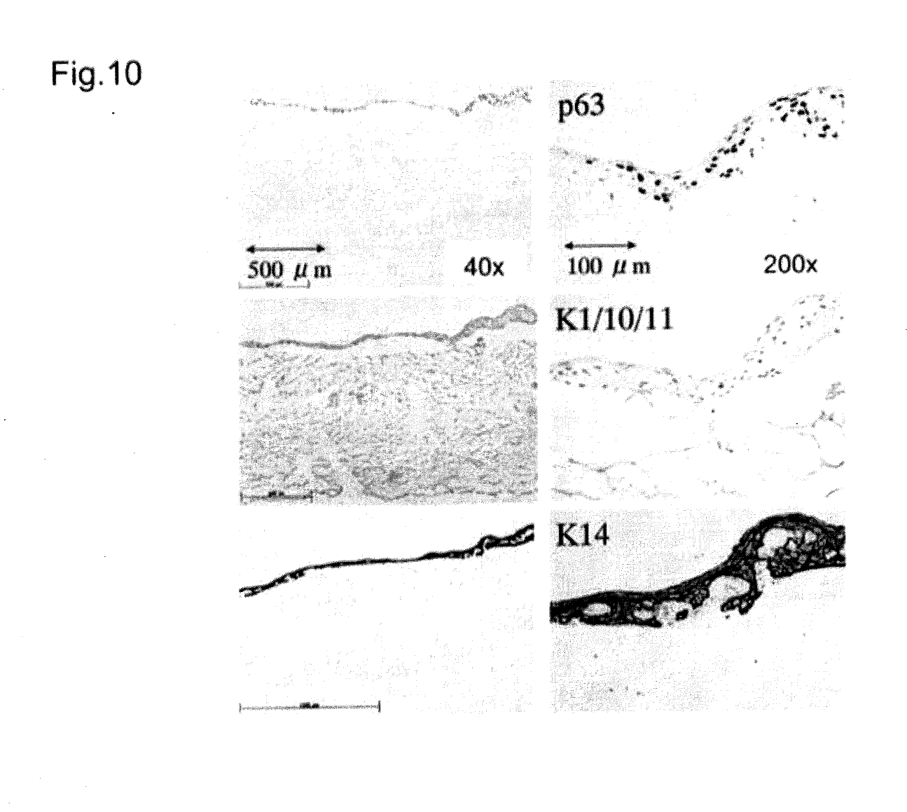

Keratinocytes in the epidermal layer positively stained with nuclear transcription factor p63, which stains cells that are undifferentiated and capable of division, and cytokeratin 14, which is a marker of basal cells; and negatively stained with cytokeratins 1/10/11, which are a marker of differentiated keratinocytes (prickle cells). These results indicate that most of the keratinocytes were basal cells that were undifferentiated and highly capable of division (FIG. 10).

Analysis

The foregoing results reveal that the fibroblasts in the peptide hydrogel not only proliferated but also expressed the function of secreting collagen as fibroblasts within the dermis.

The Examples confirmed the presence of human fibroblasts engrafting within the matrix structures in the specimens, as well as their proliferation; and also confirmed the presence of human type I collagen around the cells, revealing that the engrafting fibroblasts expressed their functions. Stratified keratinocytes were observed in the epidermal layer of the cultured skin, and the keratinocytes principally included basal cells that were undifferentiated and highly capable of division.

The foregoing results demonstrated that artificial skin that can be grafted onto a living body can be produced according to the method of the invention.

### Example 2

### Grafting of Artificial Dermis

Following the same method as described in Example 1, skin was collected from the dorsal region of male Hairless rats weighing 250 to 300 g, and fibroblasts and epidermal keratinocytes were collected and grown, thereby preparing a hybrid artificial dermal material. A 5 mm long incision was placed in the dorsal region of the rats, and subcutaneous tissue was removed to prepare a pocket. The artificial dermis was inserted into the pocket, and then the incision was sutured. The rats were divided into five groups, each group containing three rats. After embedding the artificial dermis, the embedded portions and peripheral tissue were collected from these five groups after 1, 2, 3, 4 and 5 weeks, respectively, and a histopathological evaluation was made.

### RESULTS

After embedding, blood vessels infiltrated the artificial dermis from the peripheral tissue, and inflammatory cells also infiltrated. Fibroblasts in the artificial dermis proliferated to secrete collagen, forming an extracellular matrix. The peptide hydrogel in the artificial dermis was degraded into amino acids with time, and ultimately not seen in the grafted portions.

1. A method for producing artificial skin comprising the steps of:
(A) forming a dermal layer by solidifying a mixture of dermal fibroblasts and a peptide hydrogel having a fibrous structure; and
(B) forming an epidermal layer by seeding epidermal keratinocytes onto the dermal layer obtained in Step (A), and culturing the epidermal keratinocytes.

2. The method according to claim 1, wherein the peptide hydrogel is a synthetic matrix comprising 3 to 0.1% (w/v) of amino acids and 97 to 99.9% (w/v) of water.

3. The method according to claim 1, wherein the peptide hydrogel is a synthetic matrix comprising 1 to 0.1% (w/v) of amino acids and 99 to 99.9% (w/v) of water.

4. The method according to claim 2, wherein the peptide of the peptide hydrogel is a peptide comprising 12 to 30 amino acids and having alternating hydrophobic and hydrophilic side chains, or a modified product of the peptide.

5. The method according to claim 4, wherein the amino acids are three or more types of amino acids selected from the group consisting of arginine, aspartic acid, alanine, lysine, leucine, proline, threonine, and valine.

6. The method according to claim 4, wherein the amino acids consist of arginine, asparagine, and alanine.

7. The method according to claim 4, wherein the peptide of the peptide hydrogel consists of an amino acid sequence represented by any of SEQ ID NOS. 1 to 6.

8. Artificial skin produced by the method according to claim 1.

9. The artificial skin according to claim 8, which is used for skin grafting.

10. The method according to claim 3, wherein the peptide of the peptide hydrogel is a peptide comprising 12 to 30 amino acids and having alternating hydrophobic and hydrophilic side chains, or a modified product of the peptide.

11. The method according to claim 10, wherein the amino acids are three or more types of amino acids selected from the group consisting of arginine, aspartic acid, alanine, lysine, leucine, proline, threonine, and valine.

12. The method according to claim 10, wherein the amino acids consist of arginine, asparagine, and alanine.

13. The method according to claim 10, wherein the peptide of the peptide hydrogel consists of an amino acid sequence represented by any of SEQ ID NOS. 1 to 6.

14. Artificial skin produced by the method according to claim 2.

15. Artificial skin produced by the method according to claim 3.

16. Artificial skin produced by the method according to claim 4.

17. Artificial skin produced by the method according to claim 5.

18. Artificial skin produced by the method according to claim 6.

19. Artificial skin produced by the method according to claim 7.

20. Artificial skin produced by the method according to claim 10.


SEQUENCE LISTING (6 sequences)

SEQ ID NO: 1 - protein, 16 aa, Artificial Sequence
  Synthetic sequence
RADARADARADARADA

SEQ ID NO: 2 - protein, 20 aa, Artificial Sequence
  Synthetic sequence
VKVKVKVKVPPTKVKVKVKV

SEQ ID NO: 3 - protein, 12 aa, Artificial Sequence
  Synthetic sequence
KLDLKLDLKLDL

SEQ ID NO: 4 - protein, 30 aa, Artificial Sequence
  Synthetic sequence
RADARADARADARADAGGALKRQGRTLYGF

SEQ ID NO: 5 - protein, 28 aa, Artificial Sequence
  Synthetic sequence
RADARADARADARADAGGDGRGDSVAYG

SEQ ID NO: 6 - protein, 28 aa, Artificial Sequence
  Synthetic sequence
RADARADARADARADAGPRGDSGYRGDS